(19) United States
(12) Patent Application Publication
(10) Pub. No.: US 2007/0104678 A1
(43) Pub. Date: May 10, 2007

(54) Ancient defense polymer

(76) Inventors: Michael H. May, Brantford (CA); Gary Alan Skarja, Toronto (CA)

Correspondence Address: BORDEN LADNER GERVAIS LLP, WORLD EXCHANGE PLAZA, 100 QUEEN STREET SUITE 1100, OTTAWA, ON, K1P 1J9, CA

(21) Appl. No.: 10/552,714

(22) PCT Filed: Apr. 7, 2004
(86) PCT No.: PCT/CA04/00529

§ 371 Date: Oct. 7, 2005

Related U.S. Application Data
(60) Provisional application No. 60/460,409, filed on Apr. 7, 2003.
(60) Provisional application No. 60/530,260, filed on Dec. 18, 2003.

(51) Int. Cl. A61K 31/785; C08G 63/48

(52) U.S. Cl. 424/78.27; 525/65

(57) ABSTRACT

The invention relates to an ancient defense polymer having biological activity. The polymer comprises both discrete hydrophilic blocks containing cationic charge(s) and discrete hydrophobic blocks, making it amphipathic and antimicrobial in a manner analogous to natural antimicrobial peptides that form an ancient defense mechanism employed by many organisms. Such a polymer may be formed into devices designed to resist infection or bound to the surface of an apparatus to lend the biological activity to the surface of the apparatus.

### FIELD OF THE INVENTION

The present invention relates to synthetic antimicrobial peptides, known herein as ancient defense polymers.

### BACKGROUND OF THE INVENTION

Zasloff (Nature, Vol. 415, Jan. 24, 2002, p. 389) reviewed the role of antimicrobial peptides as an evolutionary ancient, non-specific defense mechanism that is conserved throughout the plant and animal kingdoms. These peptides can rapidly act to destroy a broad range of microbes, including bacteria, fungi, viruses, and protozoa. Although the hundreds of these peptides that have been isolated in recent years display diversity in size, composition, and structure, they share several common features that are believed to confer antimicrobial activity. Namely, the antimicrobial peptides have a net positive charge, are hydrophobic and are able to form amphipathic structures (i.e. they contain clusters of hydrophobic and cationic regions that are spatially organized in discrete sectors of the molecule).

The antimicrobial peptides work by targeting a fundamental difference in the design of microbes and multi-cellular animals, best understood for bacterial targets. Bacterial targets have a negatively charged cell surface, whereas the surface of plant and animal cells has no net charge. One model proposes that the net positive charge of the peptides allows them to interact with the negatively charged bacterial cell membrane surfaces, followed by displacement of lipids, alteration of membrane structure, and in certain cases entry of the peptide into the interior of the target cell, all of which lead to cell death. In contrast, normal mammalian cell membranes are spared because they exhibit no net charge.

Since the target of the antimicrobial peptides is the microbe membrane, cell death is virtually immediate making adaptation and development of resistance difficult. In contrast, traditional antibiotics affect specific internal cell targets that leave cell morphology intact, enabling the bacteria to adapt and develop resistance.

The general nature of the antimicrobial mechanism employed by the natural peptides allows a variety of molecular sizes, structures, and compositions to exhibit activity. Therefore, analogous synthetic or semi-synthetic polymers that fulfil the basic physicochemical criteria for antimicrobial activity (i.e. cationic, hydrophobic and membrane active) are anticipated to serve as a new type of antimicrobial compound. Synthetic polymers have several distinct advantages over their natural counterparts. These include lower cost, well-defined and high purity source materials, well-developed industrial synthetic techniques, tunable degradation or resistance to degradation, and the ability to be shaped and processed into devices and products.

### SUMMARY OF THE INVENTION

It is an object of the present invention to provide a synthetic polymer analog for antimicrobial peptides.

Thus, in one aspect, the invention provides an ancient defense polymer having antimicrobial activity, the polymer comprising (i) one or more discrete hydrophobic segments, and (ii) one or more hydrophilic segments containing cationic functionality.

Further, the invention relates to a method of forming an ancient defense polymer comprising the step of forming a biologically active polymer containing a hydrophobic region or regions and a hydrophilic region or regions that carry a net cationic charge. The polymer is thus amphipathic, cationic and cell membrane-active.

Further, the invention includes an apparatus, in which the ancient defense polymer is bound in or attached to a surface of the apparatus to impart antimicrobial activity to said apparatus.

Other aspects and features of the present invention will become apparent to those ordinarily skilled in the art upon review of the following description of specific embodiments of the invention.

### BRIEF DESCRIPTION OF THE DRAWINGS

Embodiments of the present invention will now be described, by way of example only, with reference to the attached Figures, in which:

FIG. 1 represent various structures of synthetic polymers according to an aspect of the invention;

FIG. 2 represents a structure of a synthetic polymer in accordance with one aspect of the invention;

FIG. 3 illustrates the reduction in viable bacterial cell counts taken from contact area under the test copolymer films compared to controls;

FIG. 4 illustrates the reduction in viable bacterial cell counts taken from copolymer film samples compared to controls;

FIG. 5 illustrates copolymer induced hemolysis in comparison to silicone control polymer (PDMS);

FIG. 6 illustrates the reduction in viable bacterial cell counts taken from the test terpolymer films and the contact area under the test films compared to controls;

FIG. 7 illustrates the terpolymer induced hemolysis in comparison to silicone control polymer (PDMS); and

FIG. 8 represents a vehicle for delivering a synthetic polymer in accordance with the invention to a patient.

### DETAILED DESCRIPTION

Generally, the present invention provides a new type of biologically active polymer, and materials and devices formed from or incorporating the biologically active polymer.

The invention relates to an ancient defense polymer, so named because it utilizes a similar “ancient” defense as peptides possessing antimicrobial activity. The polymer has discrete hydrophilic regions containing cationic charge(s), and discrete hydrophobic regions (making it amphipathic) to affect a selected mechanism of antimicrobial activity.

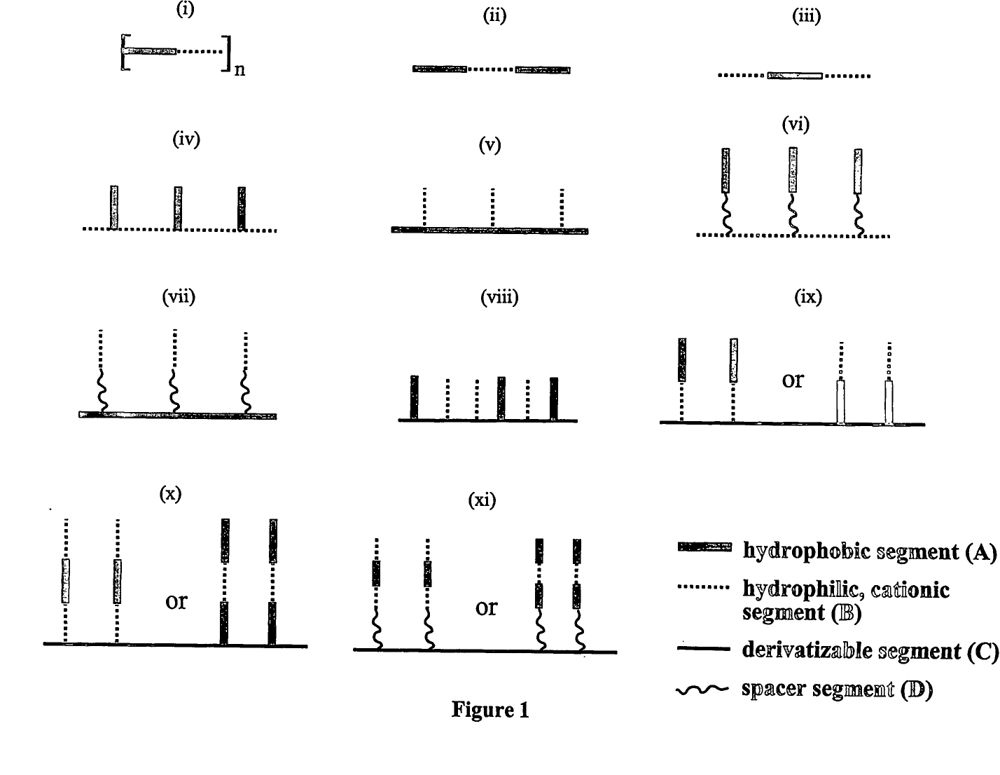

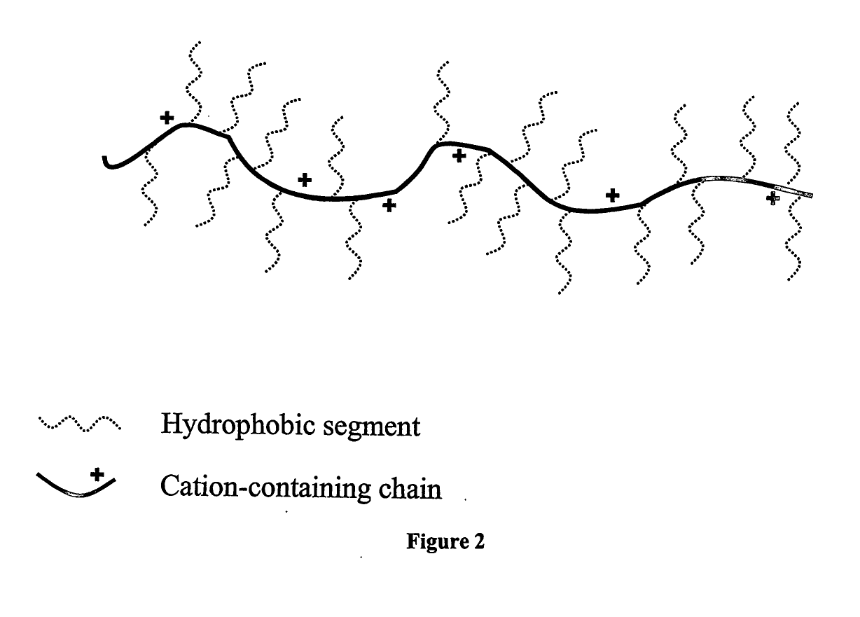

Examples of representations of such antimicrobial synthetic polymers are shown in FIGS. 1 and 2, in which segment A is hydrophobic, herein referred to as a hydrophobic segment, B represents a hydrophilic segment containing cations, and C represents a derivatizable segment, which may be degradable or non-degradable. D represents a spacer segment, which may be used to space the hydrophobic and hydrophilic segments appropriately. For example, (i)-(iii) of FIG. 1 show linear block copolymers containing hydrophobic (A) and hydrophilic cationic regions (B); (iv)-(vii) of FIG. 1 and FIG. 2 show graft polymers in which component A is grafted onto a main chain B or vice versa. The segments may be grafted onto the main chain directly or through spacer segments D. Examples (viii) to (xi) of FIG. 1 show various combinations which include the derivitizable segment C; the A and B segments may be attached to the derivatizable segment through covalent or non-covalent bonds, or through a spacer segment.

FIG. 2 shows a graft polymer containing a cation-containing backbone (B) to which is attached multiple hydrophobic segments (A). This depiction corresponds to (iv) of FIG. 1.

Exemplary blocks or regions for use as A, B and C are listed below. However, the invention is not only limited to these. Other blocks or regions which fall within the scope of A, B, and C may also be used, as would be clear to one of skill in the art.

A=a hydrophobic block (which may also be referred to herein as a segment or region) which may comprise:
1) Polymerized hydrophobic chain growth monomers. Examples of hydrophobic chain growth monomers include styrene; alkyl (meth)acrylates (e.g. C₁₋₁₈ alkyl (meth)acrylates, like methyl or butyl methacrylate), aryl (meth)acrylates (e.g. C₆₋₁₂ aryl (meth)acrylates), alkyl (meth)acrylamides (e.g. C₁₋₁₈ alkyl (meth)acrylamides, like methyl or t-butyl methacrylamide), aryl (meth)acrylamides (e.g. C₆₋₁₂ aryl (meth)acrylamides), olefins, ethers (e.g. propylene oxide), and vinyl chlorides (e.g. vinyl chloride), isobutyl vinyl ether, and methacrylonitrile;
2) Polymerized step-growth monomers. Examples of step-growth monomers include diisocyanates (e.g. 1,6 hexamethylene diisocyanate), hydrophobic diacids (e.g. sebacic acid), diamines (e.g. pentamethylene diamine), hydrophobic diacid chlorides (e.g. sebacoyl chloride), hydrophobic diols (e.g. hexamethylene glycol), and esters (e.g. ε-caprolactone); and
3) Hydrophobic (di)functional oligomers or polymers which possess reactive end groups. Reactive end groups include, for example, acid, hydroxyl, amine, isocyanate, acid chloride, ester, methacrylate, and vinyl groups. Examples included polyether diols (e.g. polypropylene oxide diol), polyester diols (e.g. polycarolactone diol), polyether methacrylates (e.g. polypropylene oxide monomethacrylate, PPO-Me) and hydroxy-terminated polybutadiene.

B=a hydrophilic block (which may also be referred to herein as a segment or region) containing cationic charge at neutral or near-neutral pH, which may comprise:
1) Polymerized cationic chain growth monomers. Examples of cationic chain growth monomers include amino (meth)acrylates (e.g. 2-(dimethylamino)ethyl methacrylate), 2-vinyl pyridine, p-N,N-dimethylamino styrene, N,N diethylallylamine, vinyl benzylamine, vinyl imidazole, and 3-aminopropyl methacrylamide hydrochloride (AMA);
2) A polymer made from a mixture of the above cationic chain growth monomers and (i) uncharged monomers that are hydrophilic (such as hydroxymethacrylates (e.g. hydroxyethyl methacrylate), vinyl acetate, and ethylene oxide) or (ii) a limited fraction (not exceeding 90% of molar composition of block B) of hydrophobic monomers, e.g. n-butyl methacrylate (BMA) and methylmethacrylate (MMA) (for additional examples, see those monomers listed under block A).
3) Cationic (di)functional oligomers or polymers which posses reactive end groups; reactive end groups include, for example, acid, hydroxyl, amine, isocyanate, acid chloride, ester, vinyl and methacrylate groups. Examples include polylysine.

C=an optional block (which may also be referred to herein as a segment or region) containing functional groups available for derivatization, which may comprise:
1) polymerized chain growth monomers containing functional groups like hydroxyl (e.g. hydroxyethyl methacrylate, polyvinyl alcohol), carboxylic acid (e.g. methacrylic acid, acrylic acid), vinyl (e.g. butadiene), acid chloride (e.g. methacryloyl chloride), and isocyanate (e.g. isocyanatoethyl methacrylate)

Blocks A and B are hydrophobic and hydrophilic blocks, respectively. These terms are used herein relatively. Thus, A must be hydrophobic relative to B, and B must be hydrophilic relative to A. Both A and B may contain various moieties some of which are hydrophilic and some of which are hydrophobic. However, on the whole A must be hydrophobic with respect to B, and vice versa. Furthermore, any one of blocks A, B, and C may contain degradable portions, to aid in degradation of the polymer in a patient.

The individual monomers listed above do not necessarily correspond to the monomers that are added to the reaction vessel for polymerization, but correspond to the monomers that make up the final polymerized product.

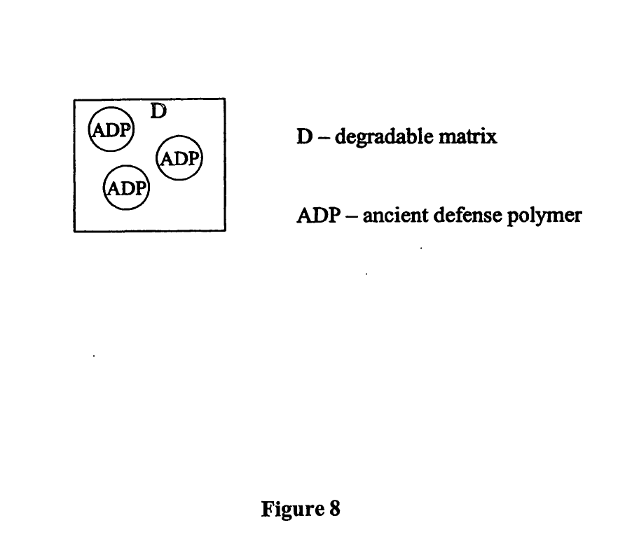

In another embodiment, the polymer of the invention may be entrapped in a degradable matrix to permit controlled release of the antimicrobial polymer. FIG. 8 illustrates a polymer according to the invention, referred to as an ancient defense polymer (ADP), placed within a degradable matrix (D). Degradable linkages (E), not shown, may be used within this polymer matrix.

The polymer(s) may be shaped or cast to generate parts of or complete devices that exhibit antibacterial activity in a number of applications including surgical devices, sterile draping and dressings, clothing, food packaging, agricultural processing and bioreactor modification. Further, such polymers may be covalently or non-covalently bound to surfaces, to lend a permanent or semi-permanent biological activity to that surface. For example, an ancient defense polymer having antimicrobial activity may be bound to a biological implant, such as a catheter, a slow-release implant, a replacement valve, a stent, or another such apparatus as may be in contact with a subject. In this way, the apparatus itself would have an antimicrobial surface, and incidence of infection during use would be reduced.

In one aspect, the invention provides an ancient defense polymer made from 1-15 mol % BMA, 549 mol % AMA, and 5090 mol % PPO-Me. 100331 In one aspect, the invention provides an ancient defense polymer of claim made from 5-50 mol % AMA, and 50-95 mol % PPO-Me.

### EXAMPLE 1

Antimicrobial Copolymers

The following antimicrobial polymers according to the invention were formulated as described herein, and possess antimicrobial properties. Copolymerization of 3-aminopropyl methacrylamide (AMA) and poly(propylene oxide) monomethacrylate (PPO-Me), both shown below, was carried out to generate a cationic, amphipathic polymer. The relative amounts of each monomer fed in the synthetic reaction was varied to generate a range of physicochemical properties.

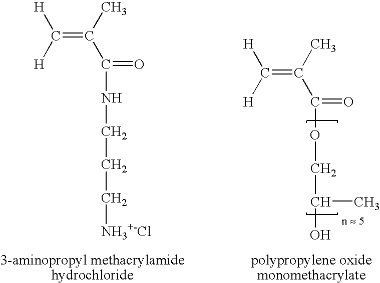

The copolymers of Example 1 can be represented by FIG. 2, in which the main chain of the polymer contains AMA and methacrylate, and the grafts contain polypropylene oxide (PPO).

Synthesis and Purification

The desired amount of each monomer was dissolved in ethanol with stirring to make up a 20% (w/v) solution. Initiator (benzoyl peroxide) was then added at 1 wt % of the total mass of monomers fed, and the solution was heated to 70° C. The reaction proceeded at 70° C. for 6 h, then the reaction solution was poured into a beaker and the solvent was dried off at room temperature. The raw polymer solid was then further dried at room temperature under vacuum for at least 3 h to remove residual solvent. The dried polymer was then extracted with a low polarity organic solvent (e.g. ethyl ether, hexane) for at least 6 h using ˜50 mL solvent per gram of polymer. The remaining polymer solid was dried and dissolved in distilled, deionized water (˜50 mL per gram of polymer). The polymer was then precipitated in 0.2 M NaOH, rinsed with distilled, deionized water and dried under vacuum at room temperature for at least 16 h. Finally, the dried polymer was dissolved in methanol, filtered to remove insoluble impurities and cast into a film.

Material Characterization

The purified polymer films were qualitatively assessed by appearance, assayed for elemental composition at Galbraith Laboratories Inc. (Knoxville, Tenn.), solution properties (lower critical solution temperature) by differential scanning calorimetry (DSC, and molecular weight by gel permeation chromatography (GPC).

Bacterial Inhibition Characterization

Evaluation of bacterial inhibition by the polymers was assayed using a modified Kirby-Bauer zone-of-inhibition test. The polymers were incubated for 24 hr in contact with a model Gram-positive bacterium (Staph. aureus) and a model Gram-negative bacterium (P. Aeruginosa). The control material was a silicone polymer. Bacteria were grown in and on sterile culture media (typically Mueller Hinton™ (MH) broth and agar) that was prepared and autoclaved prior to use. The same medium was used to prepare serial dilutions of bacteria and to plate out serial dilutions to perform viable counts.

For contact inhibition experiments, bacterial lawns were prepared by swabbing MH plates in three directions with the standardized culture. The control and test materials (in duplicate) were placed on freshly prepared bacterial lawns and the plates incubated overnight at 37° C. in a humidified incubator. The following day, the plates were examined for zones of inhibition (the dimensions of which are measured if present) and the control and test materials were carefully removed with sterile forceps. Punches of the agar containing bacteria from the contact area under each film sample were taken. The punches were vortexed in sterile saline containing sterile glass beads to dislodge the bacteria, serially diluted and viable plate counts performed. In addition, the films were removed and vortexed in sterile saline to dislodge bacteria adherent to the films and counts were performed on these as well.

Red Cell Hemolysis Assay

Blood was obtained from healthy human donors. Red cells were isolated from whole, heparinized blood by centrifugation and removal of platelet-rich plasma. The red cells were washed three times with phosphate buffered saline (PBS, 145 mM NaCl, 10 mM Na₂PO₄) and made up as a 10% (v/v) solution in PBS. Copolymer samples were placed in 1.5 mL Eppendorf centrifuge tubes (50 mg per tube) and equilibrated in 400 microlitres PBS at 37° C. for 1 h. Then 100 microlitres of the 10% red cell suspension was added to make a final red cell concentration of 2%. The copolymer samples were incubated in the red cell suspension for 1 h at 37° C. After the incubation time was complete, the red cells were centrifuged out of solution and 100 microlitres of supernatant from each sample was transferred to a 96 well plate. The absorbance of each solution was measured in a plate reader at 540 nm and compared to the positive control (0.1% Triton X-100 incubated with red cell suspension) and the negative control (PBS incubated with red cell suspension). Less than 5% hemolysis was regarded as non-toxic.

Results

The AMA monomer feed for the copolymer synthesis was varied from 10 to 50 mol % and the resulting copolymers' physical characteristics were qualitatively evaluated. Increasing AMA content resulted in increasingly stiff polymers, ranging from soft and tacky at 10 mol % AMA to semi-rigid and bendable at 37.5 mol %. The 50 mol % AMA feed copolymer was found to be water-soluble even at high pH and was therefore not characterized further. Since the 10 and 25 mol % AMA feed copolymers (90% and 75% PPO-Me, respectively) exhibited acceptable physical properties and low aqueous solubility, they were further characterized for physicochemical properties, bacterial inhibition and hemolytic potential.

Table 1 illustrates the elemental composition for the 10 and 25 mol % AMA copolymers. Elemental compositions were found to compare closely to expected, based on monomer feed ratios. In both cases, the measured nitrogen content was lower than expected indicating reduced cationic monomer incorporation in comparison to amount fed (the AMA monomer is the only nitrogen-containing species). However, the low absolute value of the nitrogen weight percent value amplifies any small differences between measured and feed values. This fact, in concert with the stated accuracy of the measurement technique (±10%) limits the precision of the calculation of polymer composition using this technique.

TABLE 1
Comparison of elemental composition measured versus
fed for 10 and 25 mol % AMA feed copolymers.

| | 25% AMA | | 10% AMA | |
|---|---|---|---|---|
| | Feed | Measured | Feed | Measured |
| | Composition | Composition | Composition | Compositio |
| Element | (wt %) | (wt %) | (wt %) | (wt %) |
| C | 60.45 | 58.57 | 60.55 | 60.36 |
| N | 2.20 | 1.76 | 0.80 | 0.60 |
| O | 27.68 | 28.32 | 29.00 | 29.60 |
| H | 9.67 | 9.29 | 9.65 | 9.44 |

Mₙ, M_{w}, and P.D. values were determined for the 10% AMA copolymer. Mₙ was 217,970; M, was 509,650, and P.D. was 2.3. This demonstrates that the product is a relatively high molecular weight copolymer (i.e. not a combination of two homopolymers).

The copolymers showed sparing solubility (≦2%) in neutral or low pH aqueous solutions at low temperature. However, in testing the solubility properties, it was discovered that the copolymers display an inverse temperature solubility profile that was reversible (i.e. they are thermoreversible gels that precipitate from solution to form a gel at differing temperatures and redissolve if cooled below the gelation temperature, T_{gel}). Table 2 provides the gelation temperatures for the 10 and 25 mol % AMA feed copolymers as analyzed by DSC.

TABLE 2
Gelation temperatures for 10 and 25 mol % AMA copolymers

Polymer Composition (mol %)

| Aminopropyl | PPO | T_{gel} |
|---|---|---|
| Methacrylamide | Methacrylate | (° C.) |
| 10 | 90 | 14.5 |
| 25 | 75 | 25.0 |

It appears that the gelation temperature increases with AMA content, probably due to a resultant increase in overall copolymer hydrophilicity. The reversibility of the gelation process was also demonstrated by cooling the samples in the DSC after heating. A dissolution exotherm was observed indicating reversibility.

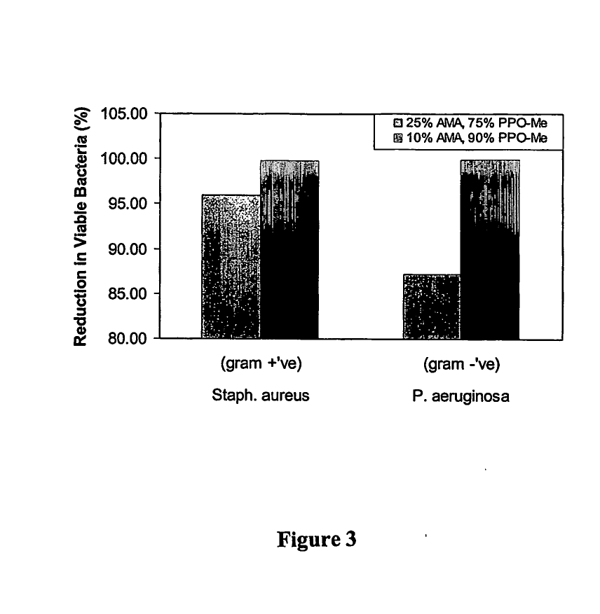

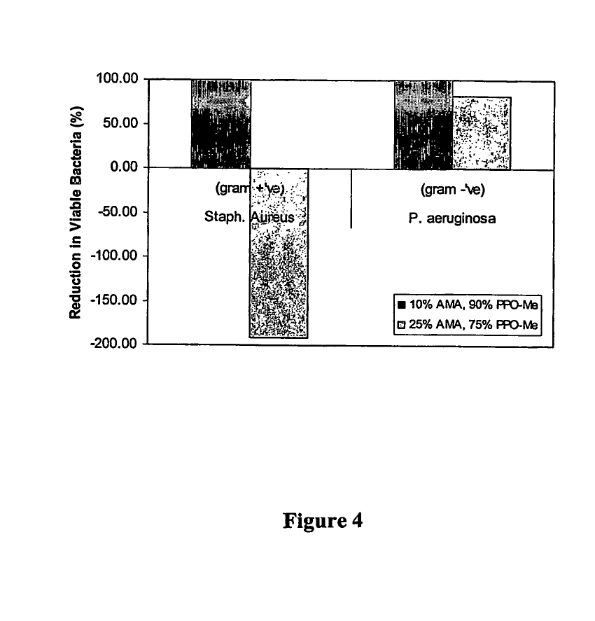

Bacterial contact inhibition results for the 25 mol % AMA and 10 mol % AMA copolymers are shown in FIG. 3. An 87 to 99.9% reduction in bacterial cell counts under the test copolymer in comparison to the silicone control indicates that both copolymers are capable of significant bacterial cell contact inhibition for both gram negative and gram positive strains. As is seen in FIG. 4, similarly high levels of reduction in bacteria adherent to the films was observed for the 10% AMA copolymer, but the 25% AMA copolymer actually shows greater (gram—′ve) bacterial adhesion than the silicone control. Therefore, the 10% AMA copolymer appears to be a more effective antimicrobial indicating that bacterial inhibitory ability is dependent on polymer composition. No zone of inhibition was observed for either copolymer suggesting that the bacterial inhibition measured was contact mediated and not a result of any material released from the film samples.

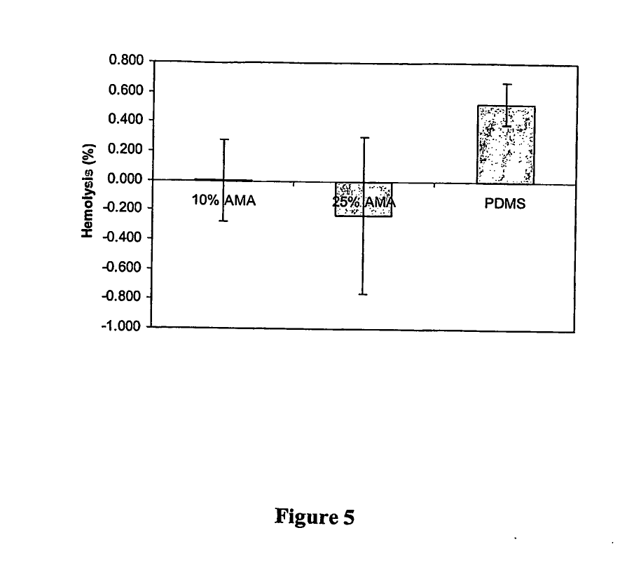

Since any effective antimicrobial must not be toxic to mammalian cells, the Applicant assayed the cytocompatibility of the antimicrobial polymers using a common human red cell hemolysis test. FIG. 5 shows the results of the hemolysis assay for the 10 and 25% AMA copolymers. Neither polymer exhibits any significant hemolysis, both results not significantly differing from 0% hemolysis (same as PBS alone). In contrast, the control silicone polymer exhibited a slight, positive hemolysis reading. This indicates that the antimicrobial polymers have a selective ability to reduce bacterial cell viability.

### EXAMPLE 2

Antimicrobial Terpolymers

In addition to the copolymers described in Example 1, terpolymers were synthesized by adding a third monomer (e.g. n-butyl methacrylate, methyl methacrylate) during polymerization to further modify resulting polymer material and bacterial inhibition properties.

The terpolymers were made as described in Example 1 using three monomers, instead of two. The purification, material characterization, bacterial inhibition characterization, and red cell hemolysis assay were performed as for Example 1.

The terpolymers of Example 2 can be represented by FIG. 2, in which the main chain of the polymer contains AMA, BMA, and methacrylate or AMA, MMA, and methacrylate, and the grafts contain PPO.

Results

The third monomer (e.g. n-butyl methacrylate, BMA and methylmethacrylate, MMA) were added at molar ratios ranging from 5 to 10% resulting in a wide variety of physical characteristics. The 10 mol % MMA terpolymer also contained 25 mol % AMA and 65 mol % PPO-Me and was found to be a clear, brittle material that showed relatively high solubility in aqueous solutions (˜2%). Therefore, since the Applicant were primarily interested in low-solubility, flexible materials, this polymer was not further characterized. Terpolymers containing BMA were found to be flexible, elastic, less water-soluble materials than corresponding MMA containing terpolymers. Therefore a 10 mol % BMA, 25 mol % AMA, 65% PPO-Me terpolymer (10% BMA) was selected for furlther characterization.

Table 3 shows the elemental composition for the 10% BMA terpolymer in comparison to the feed composition. Again, the measured composition compares closely with expected, with slightly lower than expected nitrogen which may indicate slightly lower AMA incorporation than fed.

TABLE 3
Comparison of elemental composition versus
fed for 10% BMA feed terpolymer.

| | Feed | Measured |
|---|---|---|
| | Composition | Composition |
| Element | (wt %) | (wt %) |
| C | 60.77 | 60.41 |
| O | 27.16 | 27.69 |
| H | 9.69 | 9.96 |
| N | 2.38 | 1.94 |

Similar to the copolymers described in Example 1, the 10% BMA terpolymer displays a themoreversible gelation. However, the T_{gel} for the 10% BMA terpolymer is 17.2° C., intermediate to the 10 and 25% AMA copolymers suggesting introduction of BMA as a third monomer leads to increased hydrophobicity in comparison to the copolymer of equal AMA content (25% AMA).

Mₙ, M_{w}, and P.D. values were determined for the terpolymer. Mₙ was 143,970; M_{w} was 211,930, and P.D. was 1.5. This demonstrates that the product is a relatively high molecular weight terpolymer (i.e. not a combination of homopolymers and/or copolymers).

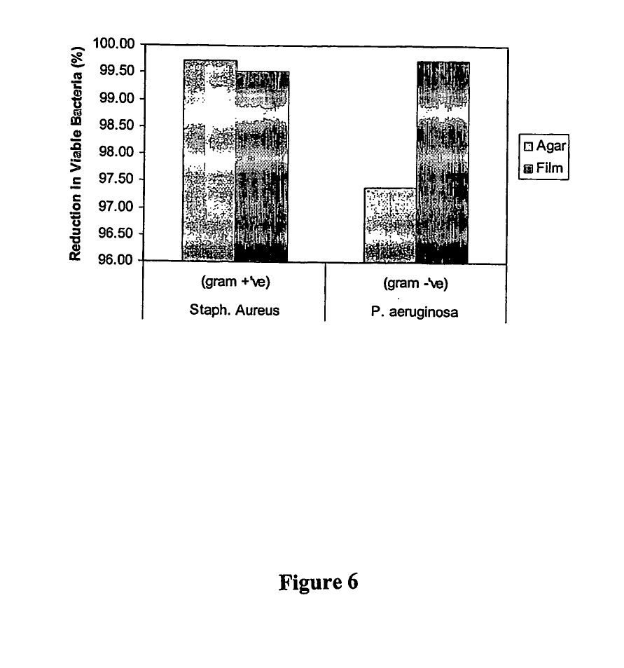

The bacterial contact inhibition results for 10% BMA are shown in FIG. 6. The film inhibits both gram positive and gram-negative bacterial growth at 97% or greater on the underlying agar and at greater than 99% on the film itself in comparison to a silicone polymer control. Again, no zone of inhibition was observed indicating that the bacterial inhibition effect was localized to the polymer surface region.

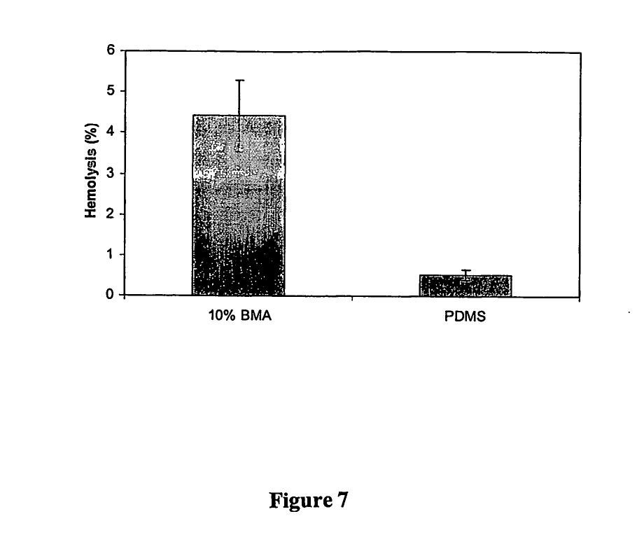

The red cell hemolysis assay was performed using the 10% BMA terpolymer to determine cytocompatibility. FIG. 7 shows the results of the hemolysis assay for 10% BMA compared to the silicone control polymer. The 10% BMA terpolymer generates a level of hemolysis that is at the threshold for toxicity indicating lesser cell compatibility than the copolymers described in Example 1.

The above-described embodiments of the present invention are intended to be examples only. Alterations, modifications and variations may be affected to the particular embodiments by those of skill in the art without departing from the scope of the invention, which is defined solely by the claims appended hereto.

1. An ancient defense polymer having antimicrobial activity, said polymer comprising one or more discrete hydrophobic segments and one or more hydrophilic segments containing cationic functionality.

2. An ancient defense polymer according to claim 1, in which said hydrophobic segment comprises:
1) polymerized hydrophobic chain growth monomers;
2) polymerized step-growth monomers; or
3) hydrophobic (di)functional oligomers or polymers.

3. An ancient defense polymer according to claim 1, in which said hydrophilic segment comprises:
1) polymerized cationic chain growth monomers;
2) a polymer made from a mixture of cationic chain growth monomers and (i) uncharged monomers that are hydrophilic or (ii) hydrophobic monomers; or
3) cationic (di)functional oligomers or polymers.

4. The ancient defense polymer of claim 1, wherein said hydrophobic segment comprises polymerized hydrophobic alkyl methacrylates, aryl methacrylates, alkyl methacrylamides, or aryl methacrylamides.

5. The ancient defense polymer of claim any one of claims 1, wherein said hydrophilic segment comprises polymerized methacrylates and/or methacrylamides.

6. The ancient defense polymer according to claim 1 comprising a copolymer of 3-aminopropyl methacrylamide (AMA,) and poly(propylene oxide)monomethacrylate (PPO-ME).

7. The ancient defense polymer according to claim 6 wherein AMA is present in an amount of from 5 to 50 mol %.

8. The ancient defense polymer according to claim 6, wherein AMA is present in an amount of about 10 mol %.

9. The ancient defense polymer according to claim 1, comprising a terpolymer of 3-aminopropyl methacrylamide (AMA,) poly(propylene oxide)monomethacrylate (PPO-ME), and methyl methacrylate.

10. The ancient defense polymer according to claim 1, comprising a terpolymer of 3-aminopropyl methacrylamide (AMA), poly(propylene oxide)monomethacrylate (PPO-ME), and n-butyl methacrylate.

11. The ancient defense polymer according to claim 1, that has a grafted chain architecture, comprising a main chain and chains grafted onto the main chain.

12. The ancient defense polymer of claim 11, wherein the main chain contains hydrophilic segments and the grafts contain the hydrophobic segments.

13. The ancient defense polymer of claim 1, wherein the hydrophilic and/or the hydrophobic segments are attached either directly or indirectly to a derivatizable polymer.

14. The ancient defense polymer according to claim 13, wherein at least one of said hydrophobic segments or said hydrophilic segments is attached to the derivatizable polymer by a spacer group.

15. The ancient defense polymer according to claim 13, wherein said hydrophobic segment and said hydrophilic segment are attached to the derivabzable polymer by a spacer group.

16. The ancient defense polymer according to claim 13, wherein at least one of said hydrophobic segments- and said hydrophilic segments is grafted onto said derivatizable polymer.

17. The ancient defense polymer according to claim 13, wherein said hydrophobic segment is grafted onto said hydrophilic segment

18. The ancient defense polymer of claim 13, wherein the derivatizable polymer comprises polymerized chain growth monomers containing reactive functional groups.

19. The ancient defense polymer of claim 18, wherein said functional groups are one or more of the groups selected hydroxyl, carboxylic acid, amine, vinyl, acid chloride, and isocyanate.

20. An apparatus comprising the ancient defense polymer of claim 1 bound in or attached to a surface of said apparatus to impart antimicrobial activity to said apparatus.

21. The apparatus of claim 20, wherein said apparatus is selected from the group consisting of an implant, a catheter, a replacement valve, a wound dressing, a medical device, and a stent.

22. The ancient defense polymer of claim 10, made from 1-15 mol % BMA, 5-49 mol % AMA, and 50-90 mol % PPO-Me.
